(19) United States
(12) Patent Application Publication
(10) Pub. No.: US 2013/0186429 A1
(43) Pub. Date: Jul. 25, 2013

(54) METHOD OF CLEANING MEDICAL INSTRUMENT AND APPARATUS THEREFOR

(71) Applicants: Kenichi Morita, Osaka (JP); Norikazu Hohshi, Osaka (JP); Kohichi Tamura, Osaka (JP)

(72) Inventors: Kenichi Morita, Osaka (JP); Norikazu Hohshi, Osaka (JP); Kohichi Tamura, Osaka (JP)

(73) Assignee: Sharp Kabushiki Kaisha, Osaka (JP)

(21) Appl. No.: 13/877,193

(22) PCT Filed: Sep. 12, 2011
(86) PCT No.: PCT/JP2011/070730

§ 371 Date: Apr. 1, 2013

(30) Foreign Application Priority Data
Nov. 25, 2010 (JP) 2010-262452

(51) Int. Cl. B08B 3/04

(52) U.S. Cl. CPC B08B 3/044
USPC 134/1; 134/184

(57) ABSTRACT

A method of cleaning a medical instrument, in which a medical instrument to which a body fluid adheres is subjected to ultrasonic cleaning in a cleaning solution in which chlorine dioxide is dissolved, and a cleaning apparatus including a cleaning tank constructed to be capable of ultrasonic cleaning, a mixing portion for generating the cleaning solution in which chlorine dioxide is dissolved, a first pipe for supplying the cleaning solution to the cleaning tank from the mixing portion, and a second pipe for supplying water to the cleaning tank, and constructed to dilute the cleaning solution supplied from the mixing portion through the first pipe with water supplied through the second pipe such that a prescribed concentration of chlorine dioxide dissolved in the cleaning solution is attained in the cleaning tank are provided.

### TITLE OF INVENTION

Method of Cleaning Medical Instrument and Apparatus Therefor

### TECHNICAL FIELD

The present invention relates to a method of cleaning a medical instrument to which a body fluid adheres and an apparatus therefor.

### BACKGROUND ART

Generally, in a large hospital having 200 to 300 or more beds, medical instruments used in surgical operations or treatments are all carried to a room called a central supply room and they are cleaned for removing such a body fluid as adhering blood. Thereafter, the medical instruments are subjected to disinfection and sterilization treatment so that they can be re-used. Though the number of medical instruments used is different depending on operation or treatment details, an amount of use thereof is great in a large hospital and medical instruments variously different in shape are used. Therefore, cleaning of medical instruments has been automated, with a large-sized cleaning machine being used instead of hand-washing. In a medium- or small-sized hospital having 200 or less beds, actually, there is a case where, rather than a large-sized cleaning machine as in a large hospital, a medium- or small-sized cleaning machine or a household dishwasher has been introduced and used, and there is also a case where medical instruments are hand-washed with the use of toothbrushes or the like.

In privately owned dental clinics, although depending on the number of patients in a day, about 100 rod-shaped dental instruments such as a scaler and a cementation instrument on average are used every day and they are cleaned each time of use. With this scale of use of medical instruments, since those medical instruments can be hand-washed with the use of toothbrushes as in a small-sized hospital, there are few cases where a cleaning machine has been introduced.

There is a report that, in spite of utmost attention, hand-washing of medical instruments after surgical operations or treatments as such has sometimes led to injuries involving blooding due to a sharp medical instrument or a pointed medical instrument, which resulted in unintended infection due to pathogenic bacteria or infectious viruses. There is also a report example that, depending on skills or physical conditions of a person in charge of hand-washing, cleanliness of cleaned medical instruments varies.

Therefore, cleaning of medical instruments including also dental instruments with the use of a cleaning machine can be regarded as very useful in terms of safety and uniformity in cleaning.

In the case of cleaning with a cleaning machine, however, depending on the number and an amount of medical instruments and how to place and arrange the medical instruments, cleanliness of cleaned medical instruments varies. Unless blood or saliva is visually recognized on cleaned medical instruments, they are determined as having been cleaned, however, pathogenic bacteria or infectious viruses may remain. Therefore, even in a hospital where a cleaning machine has been introduced, it is expected that unintended infection attributed to cleaned medical instruments occurs due to insufficient cleaning.

In addition, blood or saliva which has adhered to a used medical instrument has been known to solidify over time and removal thereof is assumed as difficult. In order to remove such solidified blood or saliva, a cleaning method making use of such a physical action as jet blast or ultrasonic vibration, a cleaning method making use of a chemical action of a dedicated cleaning solution composed of an alkali drug solution, a surfactant solution, a surface modifier solution, or other organic agent solutions, or a cleaning method combining these is applied to a recent medical instrument cleaning machine.

For example, Japanese Patent Laying-Open No. 2002-355624 (PTL 1) discloses a cleaning apparatus, in which an object to be cleaned is cleaned by injection of a cleaning solution from an injection nozzle, thereafter the cleaning solution is stored in a cleaning tank, the object to be cleaned is immersed in the cleaning solution, and thereafter the object to be cleaned is subjected to ultrasonic cleaning. Even with the cleaning apparatus disclosed in PTL 1 as such, however, in order to remove blood or saliva which has adhered to a medical instrument as being solidified, a time period for cleaning long to some extent is required.

### CITATION LIST

### Patent Literature

PTL 1: Japanese Patent Laying-Open No. 2002-355624

### SUMMARY OF INVENTION

### Technical Problem

The present invention was made to solve the problems above, and an object thereof is to provide a cleaning method and a cleaning apparatus which are capable of removing blood or saliva effectively in a short period of time and capable of eliminating as much as possible variation in cleanliness of a cleaned instrument caused by the number or an amount of instruments placed in a cleaning machine or placement and arrangement thereof, in connection with medical instruments which were used in surgical operations or treatments and to which blood or saliva solidified after having been left for a long time adheres, which is considered as difficult to remove.

### Solution to Problem

The present invention relates to a method of cleaning a medical instrument, in which a medical instrument to which a body fluid adheres is subjected to ultrasonic cleaning in a cleaning solution in which chlorine dioxide is dissolved.

In the cleaning method according to the present invention, preferably, the medical instrument to which the body fluid adheres is immersed in the cleaning solution after oscillation of ultrasonic waves in the cleaning solution contained in a cleaning tank.

In the cleaning method according to the present invention, preferably, chlorine dioxide dissolved in the cleaning solution is generated by mixing at least a chlorite aqueous solution and an activator.

In the cleaning method according to the present invention, preferably, a concentration of chlorine dioxide dissolved in the cleaning solution is raised after immersion of the medical instrument to which the body fluid adheres, and in this case, more preferably, the concentration of chlorine dioxide dissolved in the cleaning solution at the time point of immersion of the medical instrument to which the body fluid adheres is as close to 0 as possible.

The present invention also provides a cleaning apparatus for subjecting a medical instrument to which a body fluid adheres to ultrasonic cleaning in a cleaning solution in which chlorine dioxide is dissolved, which includes a cleaning tank constructed to be capable of ultrasonic cleaning, a mixing portion for generating the cleaning solution in which chlorine dioxide is dissolved, a first pipe for supplying the cleaning solution to the cleaning tank from the mixing portion, and a second pipe for supplying water to the cleaning tank, the cleaning apparatus being constructed to dilute the cleaning solution supplied from the mixing portion through the first pipe with water supplied through the second pipe such that a prescribed concentration of chlorine dioxide dissolved in the cleaning solution is attained in the cleaning tank.

### Advantageous Effects of Invention

According to the present invention, a method of cleaning a medical instrument and a cleaning apparatus therefor capable of removing such a body fluid as blood or saliva from a large number of medical instruments more quickly than in a conventional case of using an alkaline cleaning agent or a neutral cleaning agent common in cleaning of medical instruments and achieving also a remaining amount thereof equal to or less than the amount in the conventional case can be provided.

### BRIEF DESCRIPTION OF DRAWINGS

FIG. 1 is a diagram schematically showing a cleaning apparatus 1 for a medical instrument representing one preferred example of the present invention.

FIG. 2 is a diagram showing in a stepwise fashion, one example of cleaning of a medical instrument with the use of cleaning apparatus 1 shown in FIG. 1.

FIG. 3 is a diagram schematically showing one preferred example of a method of generating a cleaning solution in which chlorine dioxide is dissolved and which is used in the cleaning method according to the present invention.

FIG. 4 is a graph showing a result of measurement of an oxidation-reduction potential of a cleaning solution in which chlorine dioxide is dissolved before and after mixing of a sodium chlorite aqueous solution at a concentration of 3 to 5% containing a stabilizer and a citric acid aqueous solution at a concentration of 50%, where the ordinate represents an oxidation-reduction potential (V) and the abscissa represents time (minute).

FIG. 5 is a diagram schematically showing a sample to be cleaned 100 used for an experiment for confirming an effect of the cleaning method according to the present invention.

FIG. 6 is a diagram schematically showing how an experiment is conducted, in which sample to be cleaned 100 is immersed in a cleaning solution 200 in which chlorine dioxide is dissolved, for the purpose of removal of a blood mimicking fluid 102 from sample to be cleaned 100.

FIG. 7 is a diagram schematically showing how an experiment is conducted, in which, for the purpose of removal of blood mimicking fluid 102 from sample to be cleaned 100, an ultrasonic cleaning machine 300 for causing ultrasonic vibration of a solution in a tank is prepared, and ultrasonic cleaning machine 300 causes ultrasonic vibration while sample to be cleaned 100 is immersed in cleaning solution 200 in which chlorine dioxide is dissolved and which is contained in ultrasonic cleaning machine 300.

FIG. 8 is a diagram schematically showing a state in which blood mimicking fluid 102 has entered a gap G having a width from several ten μm to several hundred μm in a medical instrument.

FIG. 9 shows a state in which a clamp 400 to which blood mimicking fluid 102 adheres in the state in FIG. 8 is subjected to an experiment similar to that shown in FIG. 7 corresponding to the cleaning method according to the present invention.

### DESCRIPTION OF EMBODIMENTS

<Method of Cleaning Medical Instrument>

The method of cleaning a medical instrument according to the present invention is characterized by subjecting a medical instrument to which a body fluid adheres to ultrasonic cleaning in a cleaning solution in which chlorine dioxide is dissolved. The cleaning solution used in the present invention, in which chlorine dioxide is dissolved, is, for example, an aqueous solution in which chlorine dioxide called stabilized chlorine dioxide or the like is dissolved. The cleaning solution is often used in such an application as killing bacteria or viruses which adhere to medical instruments represented by an endoscope or the like having a shape complicated and difficult to clean, vegetables, fruits, fish, meat, and shells, or which are contained in water in hot springs or pools or in drinking water, that is, an application for the purpose of disinfection and sterilization, and many inventions in connection therewith have been made.

On the other hand, what is initially clarified is that the present invention is an invention of a method and an apparatus for cleaning a medical instrument which is cleaned after use in surgical operations or treatments and repeatedly used after disinfection and sterilization, but it does not mention disinfection and sterilization of medical instruments as described above.

Here, examples of “medical instruments” in the present invention include a surgical knife, a clamp, forceps, scissors, a needle, a needle holder, a retractor, a pus basin, and the like, and also encompasses such dental instruments as a scaler, a mirror, gum scissors, a sharp spoon, dental extracting forceps, an elevator, an excavator, an explorer, a plugger, and the like.

In addition, the “body fluid” which adheres to a medical instrument in the present invention refers, for example, to a liquid generated in a living body such as blood, lymph, and saliva.

According to the method of cleaning a medical instrument in the present invention, in which a medical instrument to which a body fluid adheres is subjected to ultrasonic cleaning in a cleaning solution in which chlorine dioxide is dissolved, such a body fluid as blood or saliva can be removed from a large number of medical instruments more quickly than in a conventional case of using an alkaline cleaning agent or a neutral cleaning agent common in cleaning of medical instruments and a remaining amount thereof can also be equal to or less than the amount in the conventional case.

In the cleaning method according to the present invention, preferably, the medical instrument to which the body fluid adheres is immersed in the cleaning solution after oscillation of ultrasonic waves in the cleaning solution contained in a cleaning tank. By doing so, as compared with a case where ultrasonic waves are oscillated after immersion of the medical instrument to which the body fluid adheres in the cleaning solution, advantageously, sticking of the body fluid to the medical instrument due to denaturation of protein which is a constituent element of the body fluid can be prevented and the body fluid is readily removed.

In the cleaning method according to the present invention, a frequency of ultrasonic vibration used is not particularly restricted because an effect is exhibited also in a frequency band from 5 kHz to 100 kHz as will be described later in an experimental example. In an ultrasonic oscillation apparatus represented, for example, by an ultrasonic cleaning machine for cleaning precision components, however, there are a large number of real accomplishments in adjustment of a high-frequency electric circuit for ultrasonic wave oscillation and ultrasonic waves can efficiently be transmitted to the body fluid. Therefore, a frequency band from 28 to 45 kHz is preferred.

A method of generating chlorine dioxide dissolved in the cleaning solution used in the present invention is not particularly limited, and for example, a method of generation by dissolving a gas supplied from a chlorine dioxide generation apparatus in water, a method of mixing at least a chlorite aqueous solution and an activator, and the like are exemplified. In the case of handling of a gas, however, adjustment of a concentration is difficult and the gas may be explosive depending on a concentration thereof. Therefore, chlorine dioxide is preferably generated with the method of mixing at least a chlorite aqueous solution and an activator.

In the present invention, examples of chlorites in a chlorite aqueous solution used for generation of chlorine dioxide include sodium chlorite, potassium chlorite, barium chlorite, magnesium chlorite, and the like. Since a concentration achieved of an aqueous solution in which chlorine dioxide is dissolved after mixing with an activator has substantially already been known, sodium chlorite is preferred.

In the present invention, a concentration of a chlorite aqueous solution should be adjusted such that a concentration of chlorine dioxide dissolved in an activated solution mixed with an activator attains to a defined value. In the case of setting a concentration of chlorine dioxide dissolved in a solution mixed with an activator to 20000 ppm, for example, if a chlorite aqueous solution is a sodium chlorite aqueous solution, a concentration of that aqueous solution is preferably in a range from 3 to 5%.

An activator used for generation of chlorine dioxide in the present invention means a drug having such a property that chlorine dioxide is liberated in a solution at the time when it is mixed with a chlorite aqueous solution to thereby generate an aqueous solution in which chlorine dioxide is dissolved, and for example, an organic acid represented by citric acid for adjusting pH, an inorganic acid represented by hydrochloric acid, alcohols represented by ethyl alcohol, and the like are exemplified.

Among these, with importance being placed on safety in handling, citric acid or a citric acid aqueous solution is preferably employed as an activator. In the case of using the citric acid aqueous solution as the activator, a low concentration thereof leads to a longer time required for activation, and a high concentration thereof leads to concern about re-precipitation of citric acid in a solution. Therefore, a concentration thereof is preferably within a range from 10 to 50%.

Though a ratio of mixing of a chlorite aqueous solution and an activator for generation of chlorine dioxide is not particularly restricted, for example, in the case of using a citric acid aqueous solution at a concentration of 50% as an activator with respect to 3 to 5% sodium chlorite aqueous solution, a ratio of mixing in volume is preferably within a range from 1:10 to 1:2 and more preferably within a range from 1:3 to 1:2. When a volume of a citric acid aqueous solution at a concentration of 50% is lower than a ratio of mixing in volume of 1:10, a concentration of chlorine dioxide dissolved after activation tends to be lower than a defined value. On the other hand, when a volume of the citric acid aqueous solution at a concentration of 50% is higher than a ratio of mixing in volume of 1:2, expectations that a time required for activation and a defined value for a concentration of chlorine dioxide dissolved after activation will change do not tend to be met.

In the present invention, in order to stabilize a concentration of chlorine dioxide which is liberated in water, a stabilizer is preferably added in advance to the chlorite aqueous solution used in generating chlorine dioxide. Examples of the stabilizers include 2Na₂CO₃.3H₂O₂, NaHCO₃, NaBO₃, and the like.

In the cleaning method according to the present invention, preferably, after the medical instrument to which the body fluid adheres is immersed, a concentration of chlorine dioxide dissolved in the cleaning solution is raised. As will be described later in Experimental Example 2, when a concentration of chlorine dioxide dissolved in a cleaning solution is too high, chlorine dioxide is highly oxidative. In such a case that a body fluid which adheres to a medical instrument is blood, protein components in the blood is denatured and it becomes difficult to remove the blood. Alternatively, when a concentration of chlorine dioxide dissolved in a cleaning solution is too low, there is also a possibility that a time period for cleaning necessary for sufficiently removing a body fluid which adheres to a medical instrument becomes long. Therefore, in the cleaning method according to the present invention, preferably, a medical instrument to which a body fluid adheres is immersed in a cleaning solution where a concentration of dissolved chlorine dioxide is low and thereafter the concentration of chlorine dioxide is raised, so that a time for cleaning can be shortened without denaturation of protein components in the blood. In this case, since denaturation of protein components in the blood leads to sticking of the blood to a medical instrument and removal thereof is difficult, a concentration of chlorine dioxide dissolved in a cleaning solution at the time point of immersion of the medical instrument to which the body fluid adheres is particularly preferably as close to 0 (zero) as possible.

It is noted that medical instruments used in surgical operations or treatments are various and shapes thereof range from a simple shape to a complicated shape such as a shape of a clamp. In particular, it is recognized that it takes time to remove blood or saliva which has entered a very narrow gap seen in a clamp or the like, and it has been known that cleaning is insufficient in some cases even after cleaning with a commercially available cleaning machine with the use of a general cleaning agent. As will be described later in Experimental Example 2, the cleaning method according to the present invention exhibits a cleaning effect also in various instruments having such a complicated shape.

<Apparatus for Cleaning Medical Instrument>

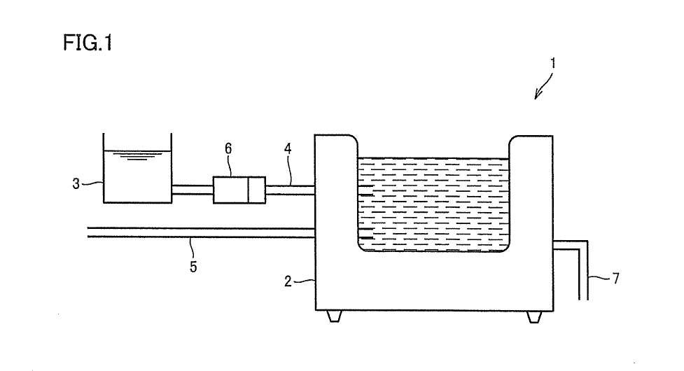

Here, FIG. 1 is a diagram schematically showing a cleaning apparatus 1 for a medical instrument representing one preferred example of the present invention. The present invention also provides an apparatus for suitably performing the method of cleaning a medical instrument according to the present invention described above. Namely, cleaning apparatus 1 for a medical instrument according to the present invention is an apparatus for subjecting a medical instrument to which a body fluid adheres to ultrasonic cleaning in a cleaning solution in which chlorine dioxide is dissolved as shown in FIG. 1, and it includes a cleaning tank 2 constructed to be capable of ultrasonic cleaning, a mixing portion 3 for generating the cleaning solution in which chlorine dioxide is dissolved, a first pipe 4 for supplying the cleaning solution to cleaning tank 2 from mixing portion 3, and a second pipe 5 for supplying water to cleaning tank 2, the apparatus being constructed such that the cleaning solution supplied from mixing portion 3 through first pipe 4 is diluted with water supplied through second pipe 5 such that a prescribed concentration of chlorine dioxide dissolved in the cleaning solution is attained in cleaning tank 2. It is noted that FIG. 1 merely shows one preferred example of the cleaning apparatus according to the present invention and the cleaning apparatus according to the present invention is not limited thereto.

In the example shown in FIG. 1, for example, in mixing portion 3, a chlorite aqueous solution to which a stabilizer has been added (for example, a sodium chlorite aqueous solution at a concentration from 3 to 5% to which 2Na₂CO₃.3H₂O₂ has been added) and a pH adjuster (for example, a citric acid aqueous solution at a concentration of 50%) are mixed to generate a cleaning solution in which chlorine dioxide is dissolved, and the cleaning solution is supplied to cleaning tank 2 of an ultrasonic cleaning machine through first pipe 4. In the example shown in FIG. 1, a delivery metering pump 6 is also provided in a part of first pipe 4, and the example is constructed such that a necessary amount of the cleaning solution generated in mixing portion 3, in which chlorine dioxide is dissolved, can be supplied to cleaning tank 2.

In the example shown in FIG. 1, cleaning tank 2 is constructed such that water is supplied through second pipe 5 and constructed such that the cleaning solution in which chlorine dioxide is dissolved and which is supplied through first pipe 4 to cleaning tank 2 is diluted to a prescribed concentration in cleaning tank 2 in cleaning of a medical instrument or a cleaned medical instrument can be rinsed with water. In addition, in the example shown in FIG. 1, a drainage pipe 7 for draining the used cleaning solution is provided in cleaning tank 2.

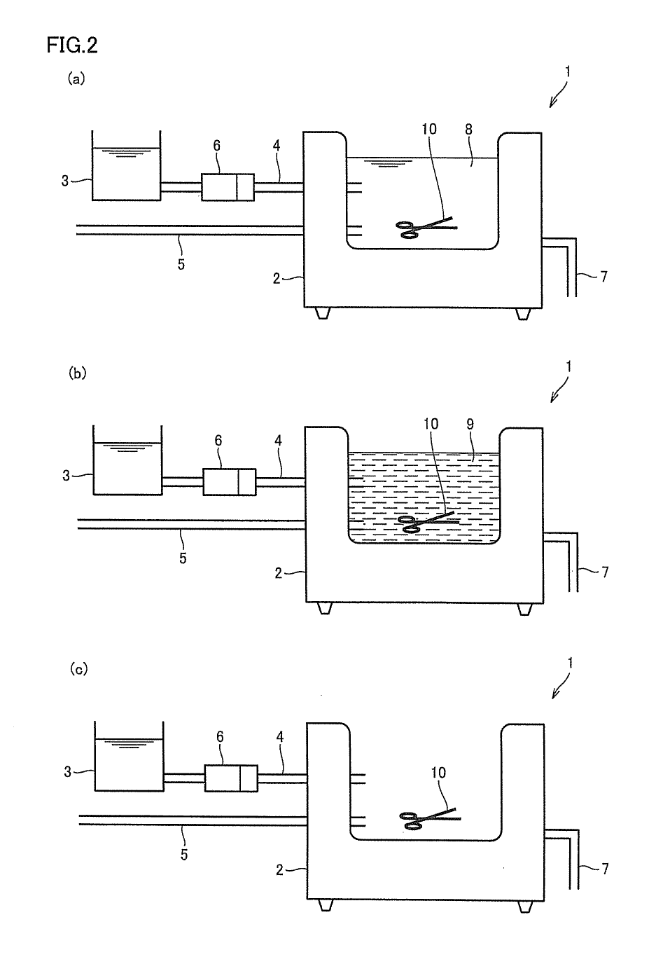

FIG. 2 is a diagram showing in a stepwise fashion, one example of cleaning of a medical instrument with the use of cleaning apparatus 1 shown in FIG. 1. Initially, FIG. 2 (a) shows a state before cleaning of the medical instrument. It is noted that FIG. 2 shows a case where a clamp 10 is cleaned by way of typical example of a medical instrument. In the state shown in FIG. 2 (a), initially, in mixing portion 3, a chlorite aqueous solution to which a stabilizer has been added (for example, a sodium chlorite aqueous solution at a concentration from 3 to 5% to which 2Na₂CO₃.3H₂O₂ has been added) and a pH adjuster (for example, a citric acid aqueous solution at a concentration of 50%) are mixed to generate a cleaning solution in which chlorine dioxide is dissolved. In order to attain a constant value for an oxidation-reduction potential, the generated cleaning solution in which chlorine dioxide is dissolved is preferably left for a certain period of time after generation, and a time for being left is preferably not shorter than 30 seconds at the minimum and more preferably not shorter than 300 seconds.

In addition, FIG. 2 (a) shows a state that a medical instrument (clamp 10) which is an object to be cleaned is arranged in cleaning tank 2 of the ultrasonic cleaning machine and water 8 is supplied into cleaning tank 2 through second pipe 5 for supply of water. This state can be expected to achieve an effect of preliminarily cleaning away such a body fluid as blood or saliva which adheres to the medical instrument by making use of the time described above during which the cleaning solution in which chlorine dioxide is dissolved is left in mixing portion 3. In addition, by applying ultrasonic vibration through ultrasonic wave oscillation within cleaning tank 2 in this state, more effective preliminary cleaning is expected.

Then, FIG. 2 (b) shows a state of cleaning of the medical instrument, in which a cleaning solution 9 in which chlorine dioxide is dissolved is present in cleaning tank 2 and a cleaning effect of removing such a body fluid as blood or saliva which adheres to the medical instrument is exhibited by application of ultrasonic vibration.

In the state shown in FIG. 2 (b), a constant amount of the cleaning solution generated in mixing portion 3 and having a constant value for an oxidation-reduction potential, in which chlorine dioxide is dissolved, is supplied into cleaning tank 2 through first pipe 4 communicating with cleaning tank 2 by using delivery metering pump 6. Thus, the cleaning solution supplied from mixing portion 3, in which chlorine dioxide is dissolved, is diluted with water in cleaning tank 2 and adjusted to cleaning solution 9 in which chlorine dioxide is dissolved at a prescribed concentration. For example, the cleaning solution is diluted such that a ratio between water and the cleaning solution generated in mixing portion 3, in which chlorine dioxide is dissolved, is 100 to 1, and thus cleaning solution 9 is adjusted.

Since water supplied into cleaning tank 2 in FIG. 2 (a) has been contaminated by preliminary cleaning before the state in FIG. 2 (b), for an improved effect of cleaning in cleaning apparatus 1, preferably, water is once drained through drainage pipe 7 and water is supplied again through second pipe 5.

Then, FIG. 2 (c) shows a state that cleaning of the medical instrument which is the object to be cleaned has ended in cleaning apparatus 1, from which the cleaning solution used for cleaning and adjusted to the prescribed concentration, in which chlorine dioxide had been dissolved, has been drained through drainage pipe 7. In this state, it is possible that a remainder of the cleaning solution used for cleaning, in which chlorine dioxide is dissolved, adheres to the medical instrument, which is not preferred as a state after cleaning. Therefore, in order to eliminate the remainder of the cleaning solution used for cleaning, in which chlorine dioxide is dissolved, in the state shown in

FIG. 2 (c), desirably, water is supplied into cleaning tank 2 through second pipe 5 to rinse the medical instrument, or preferably ultrasonic vibration is applied and the medical instrument is rinsed while water is supplied into cleaning tank 2. In this case, as water used for rinsing is drained through drainage pipe 7, cleaning with the use of cleaning apparatus 1 ends. It is noted that rinsing may be performed once, however, desirably, rinsing is performed a plurality of times so that an effect of elimination of the remainder of the cleaning solution used for cleaning, in which chlorine dioxide is dissolved, is preferably enhanced.

Though the present invention will be described in further detail with reference to experimental examples below, the present invention is not limited thereto.

### Experimental Example 1

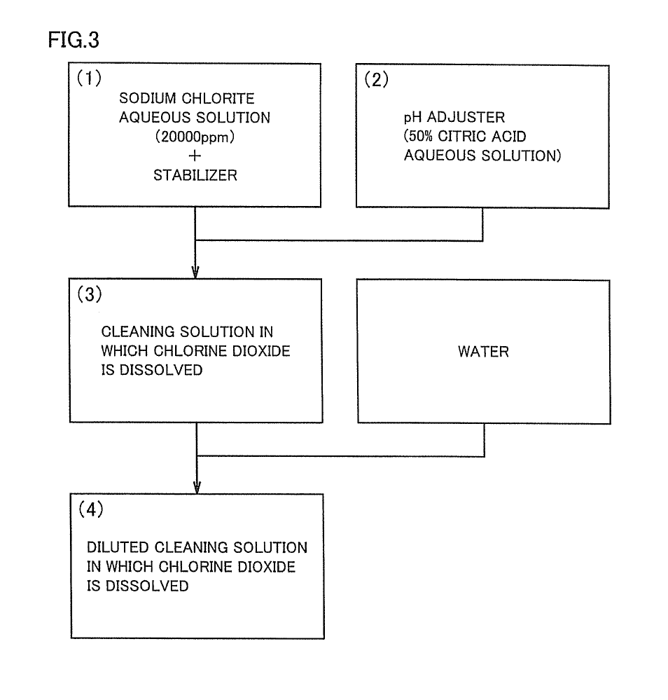

FIG. 3 is a diagram schematically showing one preferred example of a method of generating a cleaning solution in which chlorine dioxide is dissolved, to be used in the cleaning method according to the present invention. In FIG. 3, (1) represents a sodium chlorite aqueous solution at a concentration of 20000 ppm containing a stabilizer, and (2) represents a pH adjuster such as a citric acid aqueous solution at a concentration of 50%. In addition, in FIG. 3, (3) represents a solution mixture of (1) and (2) above, in which chlorine dioxide starts to be liberated, and (3) becomes the cleaning solution in which chlorine dioxide is dissolved over time. In FIG. 3, (4) represents a cleaning solution in which chlorine dioxide is dissolved, to be used for cleaning in the present invention, and it was prepared by diluting (3) above with water at a prescribed factor. Three types of the cleaning solutions (4) were set, among which an aqueous solution obtained by 40-fold dilution of (3), an aqueous solution obtained by 100-fold dilution thereof, and an aqueous solution obtained by 1000-fold dilution thereof were defined as a solution A, a solution B, and a solution C, respectively.

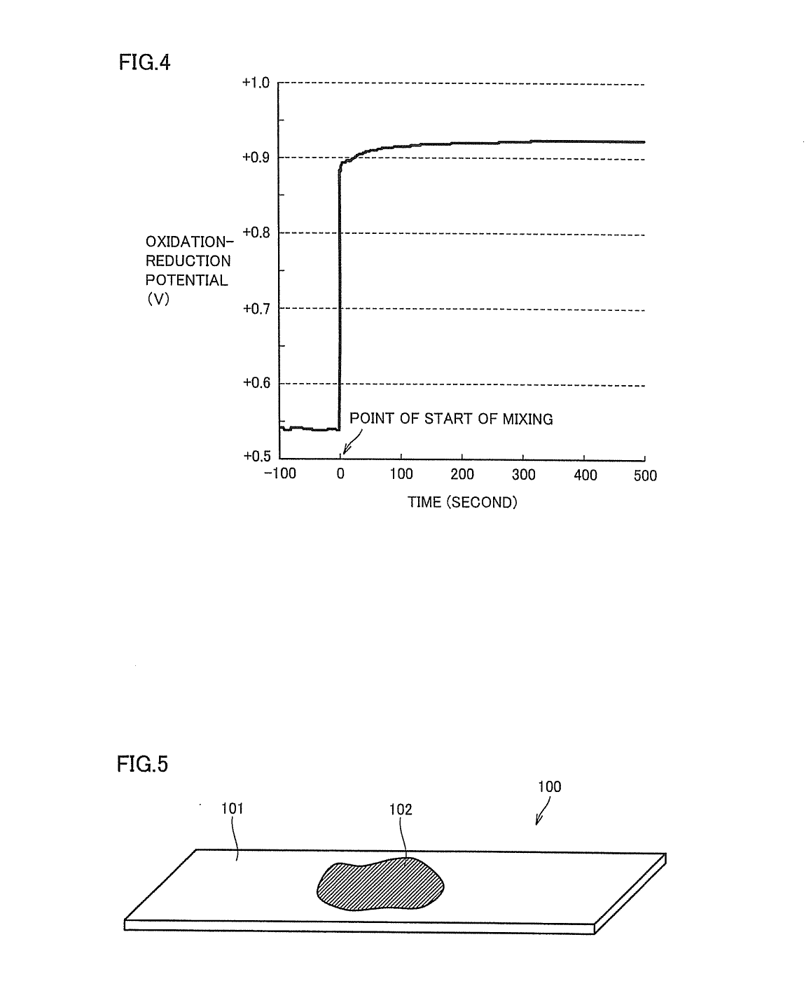

Here, FIG. 4 is a graph showing a result of measurement of an oxidation-reduction potential of the cleaning solution in which chlorine dioxide is dissolved ((3) in FIG. 3) before and after mixing of a sodium chlorite aqueous solution at a concentration from 3 to 5% containing a stabilizer ((1) in FIG. 3) and a citric acid aqueous solution at a concentration of 50% ((2) in FIG. 3), in which the ordinate represents an oxidation-reduction potential (V) and the abscissa represents time (minute). It can be seen from FIG. 4 that the oxidation-reduction potential attains to approximately +0.90 V in approximately 30 seconds after mixing and the oxidation-reduction potential attains to a constant value around +0.92 V in approximately 300 seconds. This result suggests gradual increase in chlorine dioxide liberated as a result of an action of the stabilizer contained in (1) in FIG. 3, and it can be seen that the cleaning solution in which chlorine dioxide at substantially a constant concentration is dissolved ((3) in FIG. 3) is obtained by leaving a mixture of the sodium chlorite aqueous solution containing the stabilizer and the citric acid aqueous solution after mixing, for 30 seconds or longer at the minimum and desirably for 300 seconds or longer. In order to achieve constant cleaning performance, the cleaning solution in which chlorine dioxide is dissolved, to be used in the cleaning method according to the present invention ((4) in FIG. 3), should have a constant concentration of chlorine dioxide by being left until the oxidation-reduction potential attains to +0.90 V or higher and desirably attains to a constant value around +0.92 V, and therefore solution A, solution B, and solution C described above were all diluted after being left.

FIG. 5 is a diagram schematically showing a sample to be cleaned 100, which was used for an experiment for confirming an effect of the cleaning method according to the present invention. FIG. 5 shows sample to be cleaned 100 fabricated by applying a certain amount of a blood mimicking fluid 102 in advance to a stainless plate 101 having a size of 30 mm×10 mm×1 mm and leaving the plate for solidification. Blood mimicking fluid 102 is obtained through heparin neutralization by adding protamine sulfate to heparinized sheep blood. Since sheep blood starts to be solidified after heparin neutralization, the blood mimicking fluid should be applied to stainless plate 101 immediately with a micropipet or the like. An amount of application of blood mimicking fluid 102 was adjusted such that a weight after solidification was approximately 30 mg, in consideration of an amount of blood which adheres to an actually used medical instrument. In addition, when the blood which adhered to the actual medical instrument was solidified, the blood was not removed simply by immersion in water, and similarly, blood mimicking fluid 102 was not completely removed from stainless plate 101 simply by immersion of sample to be cleaned 100 in water for approximately 30 minutes.

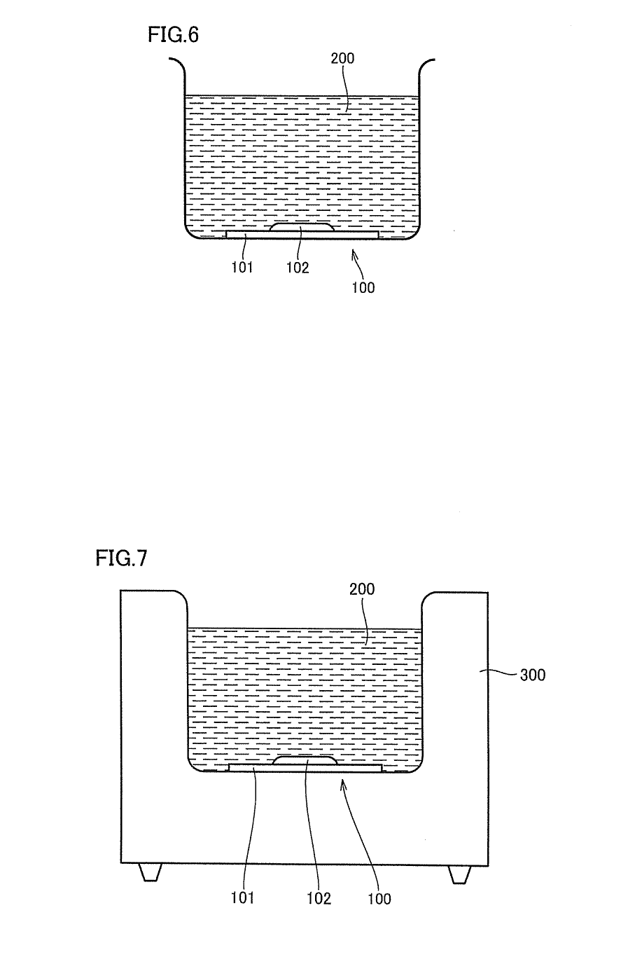

FIG. 6 is a diagram schematically showing how an experiment is conducted, in which sample to be cleaned 100 is immersed in cleaning solution 200 in which chlorine dioxide is dissolved, for the purpose of removal of blood mimicking fluid 102 from sample to be cleaned 100 described above. In addition, FIG. 7 is a diagram schematically showing how an experiment is conducted, in which, for the purpose of removal of blood mimicking fluid 102 from sample to be cleaned 100 similarly as described above, an ultrasonic cleaning machine 300 for ultrasonic vibration of a solution in a tank is prepared, and ultrasonic cleaning machine 300 causes ultrasonic vibration while sample to be cleaned 100 is immersed in cleaning solution 200 contained in ultrasonic cleaning machine 300, in which chlorine dioxide is dissolved.

In the experiment in each of FIGS. 6 and 7, three types of diluted cleaning solutions ((4) in FIG. 3) in which chlorine dioxide is dissolved, i.e., solution A, solution B, and solution C described above, were employed as cleaning solutions 200 in which chlorine dioxide is dissolved, respectively. Table 1 shows results.

TABLE 1

| | Cleaning Solution in Which | | |
|---|---|---|---|
| | Chlorine Dioxide Is Dissolved | | |
| | Solution A | Solution B | Solution C |
| Only | Not Removed in | Not Removed in | Not Removed in |
| Immersion | 30 Minutes | 30 Minutes | 30 Minutes |
| Immersion + | Completely | Completely | Completely |
| Ultrasonic | Removed | Removed | Removed |
| Vibration | in Approximately | in Approximately | in Approximately |
| | 1 Minute | 1 Minute and | 2 Minutes |
| | | 30 Seconds | |

As shown in FIG. 1, in the experiment shown in FIG. 6, even after lapse of 30 minutes since start of immersion, blood mimicking fluid 102 on sample to be cleaned 100 was not removed with any of solution A, solution B, and solution C. In contrast, in the experiment shown in FIG. 7 corresponding to the cleaning method according to the present invention, it was visually confirmed that any of solution A, solution B, and solution C substantially completely removed blood mimicking fluid 102 within 1 minute since start of ultrasonic vibration. In addition, the experiment shown in FIG. 7 was conducted under such a condition that a frequency of ultrasonic vibration ranged from 5 kHz to 100 kHz, and removal of blood mimicking fluid 102 in any frequency band was visually confirmed.

Furthermore, it was found from the results shown in Table 1 that a lower dilution factor led to a higher cleaning effect in the cleaning method according to the present invention, because the time for complete removal of blood mimicking fluid 102 was longer with solution B than with solution A and longer with solution C than with solution B in the experiment shown in FIG. 7 corresponding to the cleaning method according to the present invention. It is noted that, even with solution A, solution B, and solution C different in dilution factor, in the case of ultrasonic vibration for 5 minutes, difference in weight was not confirmed after measurement with a precision balance, of a dry weight of stainless plate 101 before and after cleaning. Therefore, it was found that, in the cleaning method according to the present invention, any of solution A, solution B, and solution C different in dilution factor could completely remove blood mimicking fluid 102 by controlling a time for cleaning.

Moreover, in the experiment shown in FIG. 7, an experiment was also conducted, in which cleaning solution 200 in which chlorine dioxide was dissolved was replaced with an inorganic alkaline cleaning agent which generally decomposes blood or saliva and then sample to be cleaned 100 was cleaned. Consequently, even after lapse of 5 minutes since start of ultrasonic vibration, a red color of blood mimicking fluid 102 was slightly visually confirmed. Therefore, it was confirmed that the cleaning method according to the present invention was higher in cleaning effect than the inorganic alkaline cleaning agent which was regarded as effective in removal of blood or saliva.

### Experimental Example 2

FIG. 8 is a diagram schematically showing a state in which blood mimicking fluid 102 has entered a gap G having a width from several ten um to several hundred pm in a medical instrument. For example, regarding such an instrument as a clamp used in a surgical operation with two plates being connected to each other at one fulcrum to function as the plates are moved, the instrument has a very narrow gap as described above, and after use thereof, a state as shown in FIG. 8 is assumed.

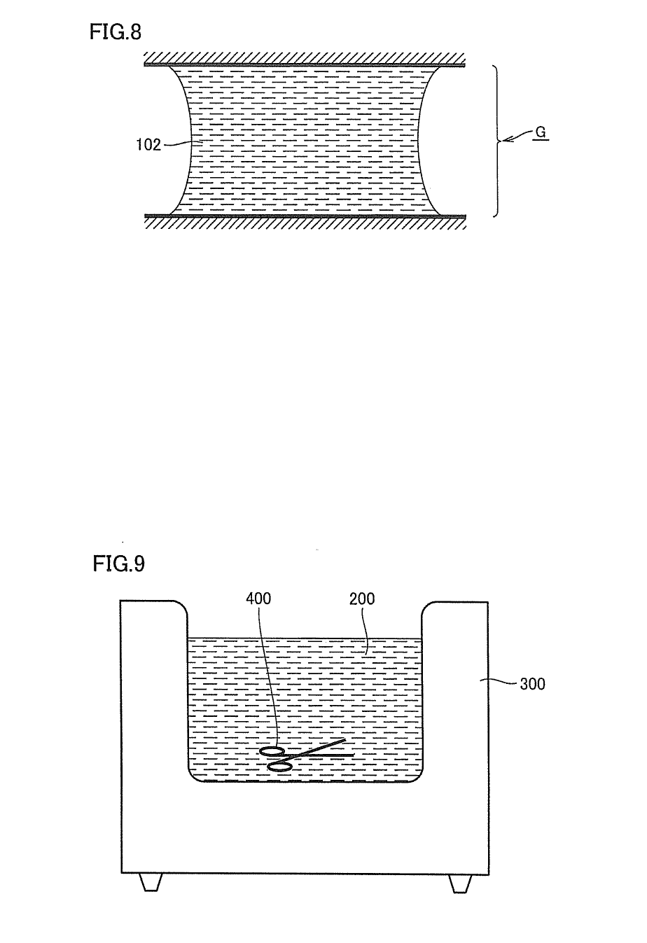

FIG. 9 shows a state in which a clamp 400 to which blood mimicking fluid 102 adheres in the state in FIG. 8 is subjected to an experiment similar to that shown in FIG. 7 corresponding to the cleaning method according to the present invention. In the experiment shown in FIG. 9, solution A or solution B described above was employed as cleaning solution 200 in which chlorine dioxide was dissolved, and blood mimicking fluid 102 of which protein components were denatured remained in gap G after lapse of 5 minutes since start of ultrasonic vibration. In contrast, when a similar experiment was conducted with the use of solution C instead of solution A or solution B, blood mimicking fluid 102 was not visually recognized in gap G. Thus, it can be seen that solution A or solution B low in dilution factor is highly oxidative and protein components are denatured earlier than decomposition of blood mimicking fluid 102, however, blood mimicking fluid 102 will be decomposed if a dilution factor is appropriate. For example, a dilution factor as in solution C could bring about even removal of blood mimicking fluid 102 which had entered gap Gas shown in FIG. 8.

In addition, a higher dilution factor of a cleaning solution in which chlorine dioxide is dissolved leads to a longer time required for cleaning. Therefore, in the case of cleaning away of blood mimicking fluid 102 which has entered gap G as shown in FIG. 8, a time required for cleaning becomes longer than in the case of cleaning of an instrument having a simple shape. From a point of view of prevention thereof, at the time of start of cleaning with the cleaning method according to the present invention, that is, at the time of start of ultrasonic vibration, preferably, a cleaning solution high in dilution factor such as solution C or water is employed as the cleaning solution in which chlorine dioxide is dissolved, and the cleaning solution not yet diluted ((3) in FIG. 3) in which chlorine dioxide is dissolved is continuously or intermittently added to the cleaning solution being used for cleaning ((4) in FIG. 3) with lapse of the cleaning time, so that the dilution factor is controlled to be lower. Thus, even blood mimicking fluid 102 which has entered gap G as shown in FIG. 8 can be removed without denaturation of protein components, and a time for cleaning can be shortened.

It should be understood that the embodiments and the experimental examples disclosed herein are illustrative and non-restrictive in every respect. The scope of the present invention is defined by the terms of the claims, rather than the description above, and is intended to include any modifications within the scope and meaning equivalent to the terms of the claims.

### REFERENCE SIGNS LIST

1 cleaning apparatus; 2 cleaning tank; 3 mixing portion; 4 first pipe; 5 second pipe; 6 delivery metering pump; 7 drainage pipe; 8 water; 9 cleaning solution in which chlorine dioxide is dissolved; 10 clamp; 100 sample to be cleaned; 101 stainless plate; 102 blood mimicking fluid; 200 cleaning solution in which chlorine dioxide is dissolved; 300 ultrasonic cleaning machine; and 400 clamp.

1. A method of cleaning a medical instrument, comprising:
subjecting a medical instrument to which a body fluid adheres to ultrasonic cleaning in a cleaning solution in which chlorine dioxide is dissolved.

2. The method according to claim 1, wherein
the medical instrument to which the body fluid adheres is immersed in the cleaning solution after oscillation of ultrasonic waves in the cleaning solution contained in a cleaning tank.

3. The method according to claim 1, wherein
chlorine dioxide dissolved in the cleaning solution is generated by mixing at least a chlorite aqueous solution and an activator.

4. The method according to claim 1, wherein
a concentration of chlorine dioxide dissolved in the cleaning solution is raised after immersion of the medical instrument to which the body fluid adheres.

5. The method according to claim 4, wherein
the concentration of chlorine dioxide dissolved in the cleaning solution at a time point of immersion of the medical instrument to which the body fluid adheres is as close to 0 as possible.

6. A cleaning apparatus for subjecting a medical instrument to which a body fluid adheres to ultrasonic cleaning in a cleaning solution in which chlorine dioxide is dissolved, comprising:
a cleaning tank constructed to be capable of ultrasonic cleaning;
a mixing portion for generating said cleaning solution in which chlorine dioxide is dissolved;
a first pipe for supplying the cleaning solution to said cleaning tank from said mixing portion; and
a second pipe for supplying water to said cleaning tank,
the cleaning apparatus being constructed to dilute the cleaning solution supplied from the mixing portion through the first pipe with water supplied through the second pipe such that a prescribed concentration of chlorine dioxide dissolved in the cleaning solution is attained in the cleaning tank.
